(19) United States
(12) Patent Application Publication
(10) Pub. No.: US 2022/0240834 A1
(43) Pub. Date: Aug. 4, 2022

(54) SYSTEM, METHOD, AND COMPUTER-ACCESSIBLE MEDIUM FOR VISUALIZATION AND ANALYSIS OF ELECTROENCEPHALOGRAM OSCILLATIONS IN THE ALPHA BAND

(71) Applicant: THE TRUSTEES OF COLUMBIA UNIVERSITY IN THE CITY OF NEW YORK, New York, NY (US)

(72) Inventors: Paul GARCIA, Irvington, NY (US); Matthias KREUZER, München (DE); Gerhard SCHNEIDER, Munich (DE); Sebastian BERGER, München (DE); David OBERT, Munich (DE)

(21) Appl. No.: 17/728,310

(22) Filed: Apr. 25, 2022

Related U.S. Application Data
(63) Continuation of application No. PCT/US2020/057370, filed on Oct. 26, 2020.
(60) Provisional application No. 62/925,650, filed on Oct. 24, 2019.

(51) Int. Cl. A61B 5/374; A61B 5/00

(52) U.S. Cl. CPC A61B 5/374; A61B 5/7264; A61B 5/4821

(57) ABSTRACT

An exemplary system, method and computer-accessible medium for providing an indication(s) to administer an anesthesia medication(s) to a patient(s) can include, for example, receiving electroencephalogram (“EEG”) information for the patient(s), determining a power spectra(s) of an alpha band of the patient(s) from the EEG information, and providing the indication(s) to administer the anesthesia medication(s) to the patient(s) based on a predetermined drop in the power spectra(s). The predetermined drop can be about 20%. An amount to assign for the predetermined drop can be received. A first derivative power spectra can be determined based on the alpha band, and the indication(s) to administer the anesthesia medication(s) to the patient(s) can be provided based on a further predetermined drop in the first derivative power spectra.

### CROSS-REFERENCE TO RELATED APPLICATION(S)

This application relates to and claims priority from U.S. Patent Application No. 62/925,650, filed on Oct. 24, 2019, the entire disclosure of which is incorporated herein by reference.

### FIELD OF THE DISCLOSURE

The present disclosure relates generally to an electroencephalogram (“EEG”), and more specifically, to exemplary embodiments of exemplary system, method, and computer-accessible medium for visualization and analysis of electroencephalogram oscillations in the alpha band.

### BACKGROUND INFORMATION

Intraoperative neuromonitoring can assist anesthesia providers to avoid administering unnecessarily high doses of anesthetics. Failure to properly titrate anesthetic medications presents a risk factor for the occurrence of perioperative neurocognitive disorders (“PNDs”). (See, e.g., References 1 and 2). PND is an umbrella term for cognitive impairment or deterioration identified in the perioperative period, and can include acute events, for example, PACU delirium, as well as delayed neurocognitive recovery after surgery. (See, e.g., Reference 3).

Previous studies have demonstrated that not only the anesthetic dose, but also the presence or absence of certain EEG patterns are correlated with PNDs. (See, e.g., References 4). In particular, pronounced alpha oscillations in the frontal EEG, especially during the emergence from anesthesia appear to be predictors of favorable neurocognitive outcomes. (See, e.g., Reference 5). The EEG alpha rhythm was originally been defined by the International Federation of Societies for Electroencephalography and Clinical Neurophysiology (“IFSECN”) as a rhythm at 8-13 Hz. (See, e.g., Reference 6). However, the classical range is often extended to group oscillations thought to be related by a common mechanism, for example, 7-17 Hz. (See, e.g., References 7-10). Although alpha oscillations over the frontal cortex in anesthesia are thought to be related to distributed, reciprocally connected, populations of cortical and thalamic neurons (see, e.g., references 11-13), the mechanisms are not fully understood. Since frontal alpha oscillations can be seen during propofol as well as volatile anesthesia it has been suggested to be a marker of a state of stable unconsciousness. (See, e.g., References 7, 8, and 14). In addition, frontal alpha power is reduced by noxious stimulation, for example, surgical incision, but can be restored by administering analgesics. (See, e.g., References 10 and 15-17). Thus, the presence of alpha oscillatory activity in the EEG can represent a state of adequate anesthesia. (See, e.g., Reference 18).

Use of the density spectral array (“DSA”) for maximization of alpha band power through adjustment of the anesthesia regimen has been suggested as an intraoperative strategy. (See, e.g., References 19 and 20). Unfortunately, elderly patients show a general decrease in EEG power under general anesthesia. (See, e.g., References 21 and 22). Furthermore, previous studies demonstrated a decrease of the peak alpha frequency with increasing age and anesthetic concentration possibly resulting in a peak frequency beyond the classic alpha range. (See, e.g., Reference 7). Visual inspection of the frontal alpha band might therefore prove challenging in certain populations or intraoperative situations. (See, e.g., References 9 and 23).

Thus, it may be beneficial to provide an exemplary system, method, and computer-accessible medium for visualization and analysis of electroencephalogram oscillations in the alpha band which can overcome at least some of the deficiencies described herein above.

### SUMMARY OF EXEMPLARY EMBODIMENTS

An exemplary system, method and computer-accessible medium for providing an indication(s) to administer an anesthesia medication(s) to a patient(s) can include, for example, receiving electroencephalogram (“EEG”) information for the patient(s), determining a power spectra(s) of an alpha band of the patient(s) from the EEG information, and providing the indication(s) to administer the anesthesia medication(s) to the patient(s) based on a predetermined drop in the power spectra(s). The predetermined drop can be about 20%. The predetermined drop can also be about 10%, about 15%, or about 25%. An amount to assign for the predetermined drop can be received.

In certain exemplary embodiments of the present disclosure, a baseline power spectra for the patient(s) can be determined. The baseline power spectra can be determined over a predetermined time series, which can be an approximate time of a medical procedure to be performed on the patient(s). The predetermined drop can be determined based on the baseline power spectra. The predetermined drop can be determined based on a rate of a drop in the power spectra(s) over time. The predetermined drop can be determined using a machine learning procedure(s), which can be a convolutional neural network.

In further exemplary embodiments of the present disclosure, a first derivative power spectra can be determined based on the alpha band, and the indication(s) to administer the anesthesia medication(s) to the patient(s) can be provided based on a further predetermined drop in the first derivative power spectra. A signal strength of the power spectra(s) can be determined, and the first derivative power spectra can be determined if the signal strength is below a threshold value. The threshold value can be received from a user(s). A finite difference approximation of the first derivative power spectra can be determined. A peak(s) in the power spectra(s) can be automatically determined. The peak(s) can be automatically determined using a linear regression procedure. A spectral property(ies) in EEG segments in the EEG information can be determined.

Additionally, exemplary system, method and computer-accessible medium for providing an indication(s) to titrate a sedation medication(s) for a patient(s) can include, for example, receiving electroencephalogram (“EEG”) information for the patient(s), determining a power spectra(s) of an alpha band of the patient(s) from the EEG information, and providing the indication(s) to titrate the sedation medication(s) for the patient(s) based on a predetermined drop in the power spectra(s). The predetermined drop can be about 20%. The predetermined drop can also be about 10%, about 15%, or about 25%. Other drop indications are possible according to various exemplary embodiments of the present disclosure. An amount to assign for the predetermined drop can be received.

In additional exemplary embodiments of the present disclosure, a baseline power spectra for the patient(s) can be determined. The baseline power spectra can be determined over a predetermined time series, which can be an approximate time of a medical procedure to be performed on the patient(s). The predetermined drop can be determined based on the baseline power spectra. The predetermined drop can be determined based on a rate of a drop in the power spectra(s) over time. The predetermined drop can be determined using a machine learning procedure(s), which can be a convolutional neural network.

In further exemplary embodiments of the present disclosure, a first derivative power spectra can be determined based on the alpha band, and the indication(s) to titrate the sedation medication(s) for the patient(s) can be provided based on a further predetermined drop in the first derivative power spectra. A signal strength of the power spectra(s) can be determined, and the first derivative power spectra can be determined if the signal strength is below a threshold value. The threshold value can be received from a user(s). A finite difference approximation of the first derivative power spectra can be determined. A peak(s) in the power spectra(s) can be automatically determined. The peak(s) can be automatically determined using a linear regression procedure. A spectral property(ies) in EEG segments in the EEG information can be determined.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary graph illustrating detected peak or no peak for patients for a standard alpha range according to an exemplary embodiment of the present disclosure;

FIG. 1B is an exemplary graph illustrating detected peak or no peak for patients for an extended alpha range according to an exemplary embodiment of the present disclosure;

FIG. 1C is an exemplary graph illustrating detected peak or no peak for patients for standard alpha wave derivative alpha range according to an exemplary embodiment of the present disclosure;

FIG. 1D is an exemplary graph illustrating detected peak or no peak for patients for an extended range alpha wave derivative range according to an exemplary embodiment of the present disclosure;

FIGS. 2A and 2B are exemplary bar graphs illustrating the number of patients with detected peaks, mixed, or no peaks according to an exemplary embodiment of the present disclosure;

FIG. 3A is an exemplary graph illustrating detected peak or no peak for patients for a power spectral density of an EEG according to an exemplary embodiment of the present disclosure;

FIG. 3B is an exemplary graph illustrating detected peak or no peak for patients for the first derivative of the power spectral density of an EEG according to an exemplary embodiment of the present disclosure;

FIG. 4A is an exemplary boxplot illustrating centroid frequencies of the EEG from patients where a peak was detected, with ‘mixed results’, or ‘no peak’ detected for the standard alpha range according to an exemplary embodiment of the present disclosure;

FIG. 4B is an exemplary boxplot illustrating centroid frequencies of the EEG from patients where a peak was detected, ‘with mixed results’, or ‘no peak’ detected performed using a power spectral density according to an exemplary embodiment of the present disclosure;

FIG. 5A is an exemplary boxplot illustrating results from the evaluation of an alpha oscillatory power of patients with peaks derived from the power spectral density according to an exemplary embodiment of the present disclosure;

FIG. 5B is an exemplary graph illustrating cumulative distribution of cases with a peak above a defined, stepwise, increasing dB threshold according to an exemplary embodiment of the present disclosure;

FIG. 6A-6C are exemplary spectral diagrams and graphs for density spectral array and power spectral density for three exemplary cases according to an exemplary embodiment of the present disclosure;

FIG. 7A is an exemplary spectral diagram illustrating increasing 10 Hz amplitude in noise shown for the power spectral density according to an exemplary embodiment of the present disclosure;

FIG. 7B is an exemplary spectral diagram illustrating increasing 10 Hz amplitude in noise shown for the first derivative of the power spectral density according to an exemplary embodiment of the present disclosure;

FIG. 8A is an exemplary spectral diagram illustrating accelerating alpha in noise for the power spectral density according to an exemplary embodiment of the present disclosure;

FIG. 8B is an exemplary spectral diagram illustrating accelerating alpha in noise for the first derivate of the power spectral density according to an exemplary embodiment of the present disclosure;

FIG. 9 is an exemplary graph illustrating the effect of discrete differentiation on EEG in the time and frequency domains according to an exemplary embodiment of the present disclosure;

FIGS. 10A-10C are exemplary spectrograms according to an exemplary embodiment of the present disclosure;

FIG. 11 is an exemplary flow diagram of a method for providing an indication to administer an anesthesia medication to a patient according to an exemplary embodiment of the present disclosure;

FIG. 12 is a set of illustration of exemplary frontal EEG patterns of two patients illustrating varying degrees of discontinuous, low-voltage activity according to an exemplary embodiment of the present disclosure;

FIG. 13 is a set of illustration of exemplary distinct frontal EEG patterns from two patients illustrating activity in the low and moderate frequency range according to an exemplary embodiment of the present disclosure;

FIG. 14 is an exemplary flow diagram of a method for providing an indication to titrate a sedation medication for a patient according to an exemplary embodiment of the present disclosure; and

FIG. 15 is an illustration of an exemplary block diagram of an exemplary system in accordance with certain exemplary embodiments of the present disclosure;

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

### Exemplary Methods

### Exemplary EEG Recording

The exemplary system, method and computer-accessible medium, according to an exemplary embodiment of the present disclosure, was used to analyze EEG records from 180 patients undergoing non-cardiac, non-neurologic surgery under general anesthesia. For EEG recording, either a Bispectral Index (“BIS” with a sampling rate of 128/s) or Entropy (GE Healthcare, Helsinki, Finland, with a sampling rate: 100/s) anesthetic depth monitor was used. To ensure comparability, raw EEG from the BIS was resampled to about 100 Hz (plus or minus about 10%). For the exemplary analysis, ten seconds of artefact-free, non-burst-suppression EEG, were used. To exclude the influence of surgical stimulation, EEG data recorded two to five minutes prior to surgical incision was selected.

### Exemplary EEG Analytical Approach

The power spectrum of the EEG in a double-logarithmic presentation can coarsely follow a 1/f^{k} distribution. The k-coefficient can lie around k=1 at wakeful eyes open or eyes closed conditions, and can decrease to around k=2 during unconscious conditions with propofol or xenon. (See, e.g., Reference 24). The EEG power spectrum can include a 1/f background component and additional oscillatory activity on top thereof. (See, e.g., References 24 and 25).

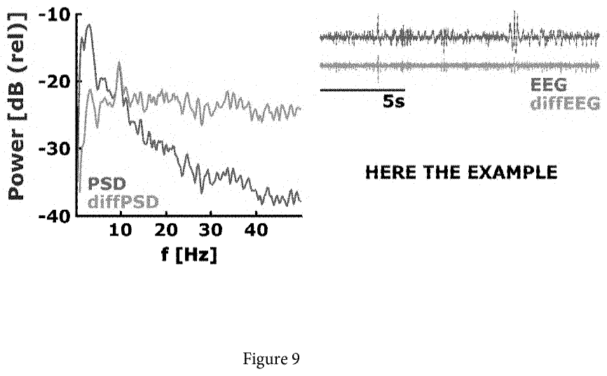

Differentiation can function as a ‘whitening’ filter on signals with a 1/f spectral distribution. The exemplary discrete-time differentiation approach can compensate for the 1/f^{k} low pass characteristic of the EEG. (See, e.g., FIG. 9). For example, it can generate a horizontal background component with the oscillatory components presented as peaks. A differentiation of an EEG signal can be performed by using the finite difference approximation for the first derivative. The forward difference approach, for example, the difference of the amplitude values x(t₂)−x(t₁) with t₂>t₁, was used. (26). If log PSD(f)=1/f, then the product of power (log PSD) and frequency (f) can be constant for each f. Using the finite difference sequences that can cause the whitening can have the same effect. In terms of oscillatory activity, this exemplary procedure of approximating the derivative can indicate that higher-frequency oscillatory components (e.g., here alpha oscillations on top of slow delta activity) can become dominant. (See, e.g., Reference 27).

For the exemplary analyses, the finite difference sequence (“diffEEG”) of each of the 10 s EEG episodes was obtained by using the MATLAB R2017a (The MathWorks Inc., Natick, Mass.) diff function. For a given time series X=[x(t1), x(t2), . . . , x(tn)] of n samples, its finite difference sequence X′=[x(t2)−x(t1), . . . , x(tn)−x(tn−1)] can include n−1 samples.

The power spectral density (“PSD”) was determined using Welch's power spectral density estimate for the 10 s EEG episodes as well as for the diffEEG of these episodes (“diffPSD”). MATLAB R2017a (The MathWorks Inc., Natick, Mass.) pwelch function (e.g., default settings and NFFT=128) was utilized. A finite difference sequence is a discrete-time approximation of the first derivative dx(t)/dt of a signal x(t).

### Exemplary Automated Peak Detection

For the automated peak detection, a linear regression procedure was used for the PSD, and a mean and standard deviation approach was used for the diffPSD. The first procedure was to calculate the PSD and diffPSD for the 10 s EEG episode of each patient.

For peak detection in the PSD, the linear fit of the PSD was calculated in a logarithmic scale using the MATLAB polyfit function. The range for calculation of the fit was limited to frequencies below about 30 Hz (plus or minus about 10%). The classical alpha range from 8-12 Hz, or the extended alpha range from 7-17 Hz, was ignored for fitting, because peaks in this range can influence the fit. (See. e.g., References 7 and 10. The influence of the choice of the alpha range on the peak detection was evaluated by a stepwise increase on the alpha range to be excluded. In order to evaluate the influence of the power in the delta range on the fit, and consequently on the peak detection, the frequencies from about 4 Hz downwards (plus or minus about 10%) were excluded in a stepwise manner.

The polyfit function can also return, for example, a 95% prediction interval, and the occurrence of a peak was defined as at least one value in the classical or extended alpha range of the PSD being above the limits of the prediction interval.

For the diffPSD, the mean power and standard deviation of the frequencies were calculated up to about 30 Hz (e.g., plus or minus up to about 10%), with the power values in the classical or extended alpha range excluded. The power in the delta range was excluded in a stepwise fashion and evaluated the influence of a stepwise increasing alpha range as well. Similar to the prediction interval for the PSD, a peak of the diff PSD if the power in the alpha range was above the calculated mean plus two times the standard deviation was defined.

### Estimation of the Centroid Frequency

In order to supplement the information regarding a peak, a parameter was added that can reflect the spectral properties of the EEG segments. The EEG was filtered to about the 0.5 to 30 Hz range (e.g., plus or minus about 10%) using the MATLAB filtfilt function and a 5th order Butterworth filter. The centroid frequency of the filtered EEG was estimated for each of the 180 cases. In order to approximate the centroid frequency the zero-crossing-rate was evaluated. (See. e.g., Reference 28. These exemplary calculations were performed with MATLAB. Using this exemplary procedure, a qualitative component was added to the ‘peak detected’ or ‘peak not detected’ decision.

### Exemplary Estimation of Peak Strength

The oscillatory alpha power, defined as the difference between the maximal power in the defined alpha range and the (i) upper bound of the prediction interval of the linear regression PSD or (ii) the mean and 2-fold standard deviation diffPSD, was calculated.

### Exemplary Statistical Analyses

### Automated Exemplary Peak Detection

The number of alpha peaks detected by PSD and diffPSD including the delta range was calculated, and the calculation was repeated excluding the delta range. Additionally, cases in which the gradual exclusion of the delta range influenced the peak detection were identified. These cases were classified as ‘mixed’. To evaluate the influence of the alpha range on peak detection, the alpha range was dynamically extended from about 12 Hz (e.g., plus or minus up to about 10%) to 17 Hz (e.g., plus or minus up to about 10%).

### Exemplary Evaluation of the Centroid Frequency

The centroid frequencies in the groups ‘no peak’, ‘mixed’, and ‘peak’ for automated detection using the Mann-Whitney U test were compared, and the area under the curve (“AUC”) with 95% confidence intervals were derived from 10 k-fold bootstrapping as effect size. A MATLAB-based MES toolbox was utilized. (See, e.g., Reference 30). The AUC can be used to evaluate the strength of an effect and helps to balance out unclear results derived using the p-value alone. (See, e.g., Reference 31).

### Evaluation of the Oscillatory Alpha Power

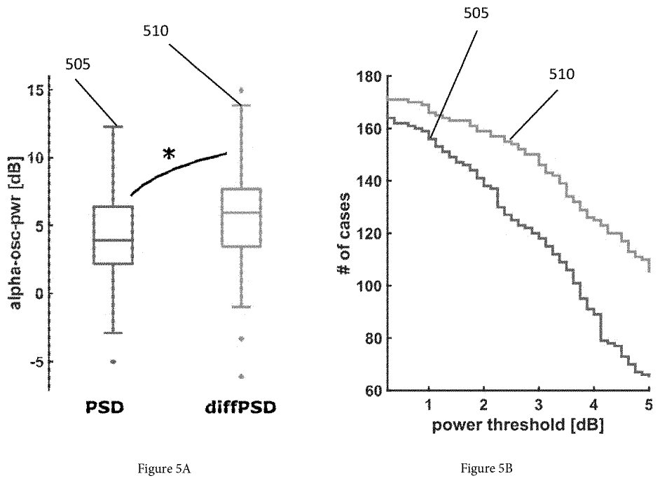

FIG. 5A shows an exemplary boxplot illustrating results from the evaluation of an alpha oscillatory power of patients with peaks derived from the power spectral density according to an exemplary embodiment of the present disclosure. In particular, FIG. 5A shows boxplots together with the AUC for the alpha-oscillatory power in dB of the cases with a peak for the PSD and diffPSD. The MATLAB stairs function was used to highlight the distribution of oscillatory alpha power over the cases with a peak. In order to find significant differences in the distribution of the peaks, the two-sample Kolmogorov-Smimov goodness-of-fit hypothesis test was applied.

### Exemplary Cases for Alpha Peak Visualization

Three exemplary cases that highlight the capability of the exemplary system, method, and computer-accessible medium to highlight the alpha peak oscillations on a monitor screen without running into the scaling issues. (See, e.g., Reference 20). The data of three cases that were recorded from patients included in a study previously published. (See, e.g., Reference 5). The EEG was originally recorded with 250 Hz using a SEDLine Legacy device. Prior to processing, band-pass filters were applied to the EEG to a range from 0.5 to 47 Hz (e.g., 5th order Butterworth, MATLAB filtfilt), followed by a downsampling to 125 Hz. The density spectral array (“DSA”) was constructed by calculating the PSD (e.g., Welch's method, MATLAB pwelch) for 10 s of EEG with a one second shift and a frequency resolution of 0.244 Hz.

### Exemplary Results

### Automated Exemplary Peak Detection

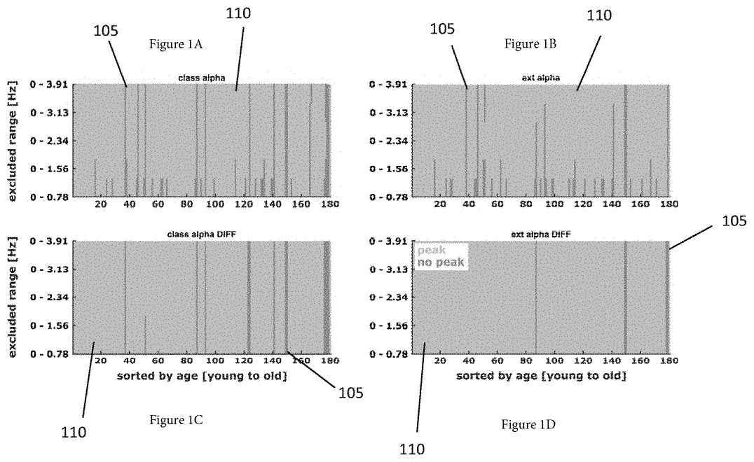

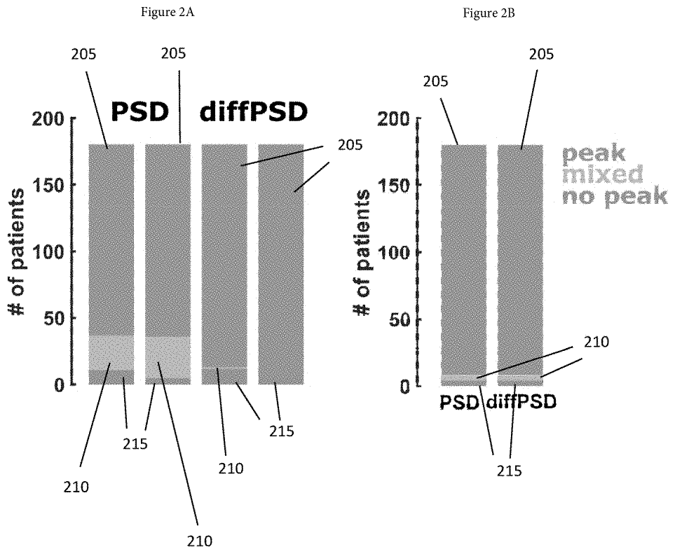

The analyses of the PSD and diffPSD indicated a more robust behavior of the diffPSD approach for automated peak detection that was not dependent on the range of excluded frequencies in the delta-range. The graphs shown in FIGS. 1A-1D, and the stacked bar plots shown in FIGS. 2A and 2B illustrate the exemplary details of the peak/no peak analysis. For the PSD analysis, the number of no peaks was significantly higher when frequencies from about 0.78 to 3.91 Hz (plus or minus about 10%) were included in the fit than if they were excluded. For the classical alpha excluded range, 36 ‘no peaks’ were found when the about 0.78 to 3.91 range (e.g., plus or minus up to about 10%) was included vs. 13 ‘no peaks’ when this range was excluded (e.g., p=0.001; Chi-Square=11.43, FIG. 1A). For the extended alpha range excluded, 36 ‘no peaks’ were found when the about 0.78 to 3.91 range (e.g., plus or minus up to about 10%) was included vs 6 ‘no peaks’ when this range was excluded (e.g., p<0.001; Chi-Square=22.67, FIG. 1B).

No significant difference in the diffPSD analyses were observed. For the classical alpha band excluded, the number of ‘no peaks’ was 13 (e.g., with 0.78-3.91 Hz) vs. 12 without the range (e.g., p=1; Chi-Square=0, FIG. 1C). For the extended alpha range excluded, w 5 ‘no peaks’ for both settings (e.g., p=1; Chi-Square=0, FIG. 1D) were found.

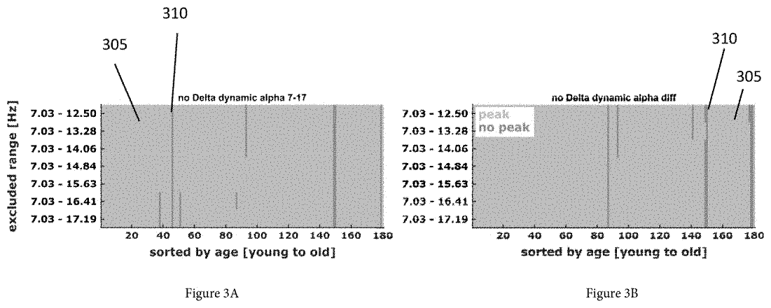

Further, a lower number of patients with a ‘mixed’ result in the diffPSD group was found. For example, whether a peak was detected or not, was independent of the stepwise procedure of excluding the frequencies. This result can indicate that the diffPSD approach may not be influenced by the choice of including or excluding the delta range for alpha peak detection. 11 ‘no peaks’, 26 ‘mixed’, and 143 ‘peaks’ were located when using the classical alpha range and the PSD approach and 12 ‘no peaks’, 1 ‘mixed’ and 167 ‘peaks’ when using diffPSD (e.g., p<0.001; Chi-Square=26.40). For the extended alpha range it was 5/31/144 PSD vs. 5/0/175 diffPSD (e.g., p<0.001; Chi-Square=34.01). FIGS. 2A and 2B show exemplary bar graphs illustrating the number of patients with detected peaks, mixed, or no peaks according to an exemplary embodiment of the present disclosure. The definition of the alpha range influenced both peak detection approaches. For these analyses, the delta range was excluded, which was based on the results presented above, which are illustrated in the graphs shown in FIGS. 3A and 3B. With the delta range excluded, no significant difference between the PSD and diffPSD was observed. Both exemplary approaches showed 4 ‘no peaks’, 4 ‘mixed’ peaks, and 172 ‘peaks’.

FIGS. 1A-1D show exemplary graphs providing exemplary information regarding a detected peak (e.g., identified by element number 105)) or no peak (e.g., identified by element number 110) for the 180 patients along the x-axis. The y-axis shown in FIGS. 1A-1D indicate the range that was excluded for the linear fit of the PSD, or the mean and standard deviation calculation for the diff PSD. The range of the excluded frequencies strongly influenced the peak detection of the PSD when looking for a peak in the standard alpha range (see e.g., FIG. 1A) and the extended alpha range. (See e.g., FIG. 1B). The vertical lines that do not cross the entire plot indicate that for this patient whether or not a peak was detected, dependent on the setting. For the diffPSD less peaks were detected for the classical alpha range (see e.g., FIG. 1C) than for the extended range. (See e.g., FIG. 1D). However, the peaks were detected independent of the excluded frequency range.

FIGS. 2A and 2B illustrate Information regarding the number of patients with a detected peak (e.g., areas 205), a peak that was detected depending on the range of excluded frequencies in the delta range (e.g., areas 210), or no peak detected at all (e.g., areas 215).

FIGS. 3A and 3B illustrate information regarding a detected peak (e.g., identified by element number 305) or no peak (e.g., identified by element number 310) for the 180 patients along the x-axis. The y-axis indicates the range that was excluded for the linear fit of the PSD, or the mean and standard deviation calculation for the diff PSD. For both, the PSD (see e.g., FIG. 3A) as well as the diffPSD the selection of the excluded range influenced the peak detection as marked by the ‘broken lines’.

### Exemplary Centroid Frequencies

Significantly different centroid frequencies were found for the ‘peak’, ‘mixed’ peak, or ‘no peak’ decisions for both the exemplary PSD approaches (e.g., Kruskal-Wallis: p<0.001; Chi-square: 24.48) and the exemplary diffPSD approach (e.g., p=0.001; Chi-square: 14.41) when excluding the classical alpha range. For the exemplary PSD approach, the median centroid frequencies were 15.3 Hz (e.g., IQR: 2.3 Hz) for ‘no peak’, 13.9 (1.9) Hz for ‘mixed’, and 12.5 (1.8) Hz for ‘peak’. Consequently, post-hoc analysis revealed a significant difference between the ‘peak’ and ‘mixed peak’ (e.g., p<0.001) as well as ‘peak’ and ‘no peak’ (e.g., p=0.001) group. There was no significant difference between the ‘mixed peak’ and ‘no peak’ patients (e.g., p=0.498). For the exemplary diffPSD, the median frequencies (e.g., and IQR) were 15.6 (3.7) Hz for the ‘no peak’, 14.6 Hz for the one ‘mixed’ case and 12.7 (1.8) Hz for the ‘peak’ cases. Because only one case with a ‘mixed peak’ was observed, no post hoc analyses was performed, and the Mann-Whitney U test was presented together with the AUC for the comparison ‘no peak’ vs. ‘peak’. The centroid frequency was significantly higher in the ‘no peak’ cases (e.g., p<0.001) and the AUC indicated a strong effect AUC=0.81 [0.64 0.95].

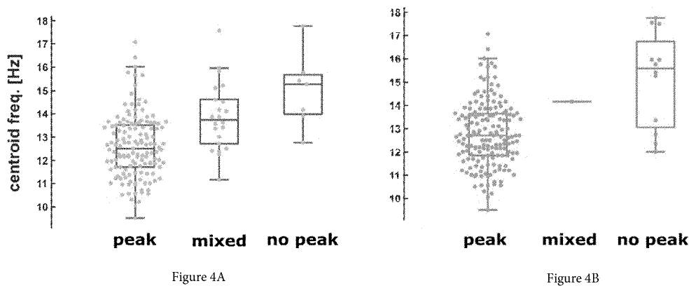

FIGS. 4A and 4B illustrate boxplots representing the centroid frequencies of the EEG from patients where a peak was detected, with ‘mixed results’, or where ‘no peak’ was detected for the classical alpha range. The boxplot shown in FIG. 4A illustrates peak detection performed with the PSD and the boxplot shown in FIG. 4B illustrates peak detection performed with the diff PSD.

### Exemplary Oscillatory Alpha Power

For the exemplary analyses, the ‘delta range’ and ‘classical alpha’ excluded setting was used. A significant difference was observed between the alpha-oscillatory power in the exemplary PSD approach (e.g., median: 3.93 dB, IQR: 4.22 dB) and the diffPSD approach (e.g., median: 5.98 dB, IQR: 4.22 dB) as depicted by the AUC=0.64 and the 95% confidence interval from 0.58 to 0.70. FIGS. 1A-1D illustrate the corresponding box plot as well as the stairs plot. The stairs plot depicts the significantly different distribution of oscillatory alpha power for the PSD and diffPSD approach (e.g., p=0.022).

FIGS. 5A and 5B illustrate graphs providing exemplary results from the evaluation of the oscillatory power. In particular, FIG. 5A shows an exemplary graph of boxplots representing the alpha oscillatory power of patients with peaks as derived from the PSD (e.g., element 505) and diffPSD (e.g., element 510) approach. FIG. 5B shows an exemplary graph providing an exemplary cumulative distribution of cases with a peak above a defined, stepwise increasing dB threshold.

### Exemplary Cases Presenting the Visual Highlighting of the Alpha Oscillation

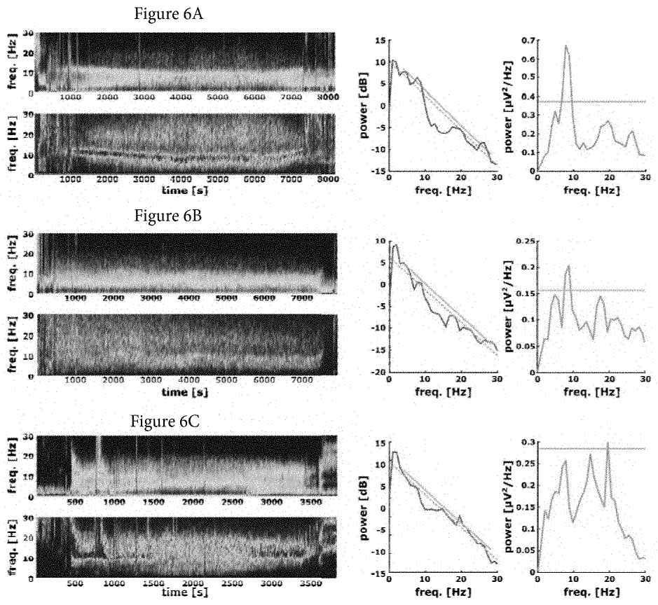

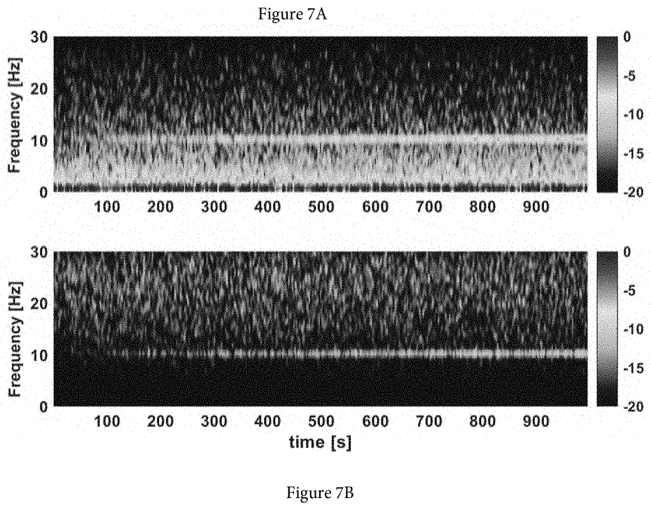

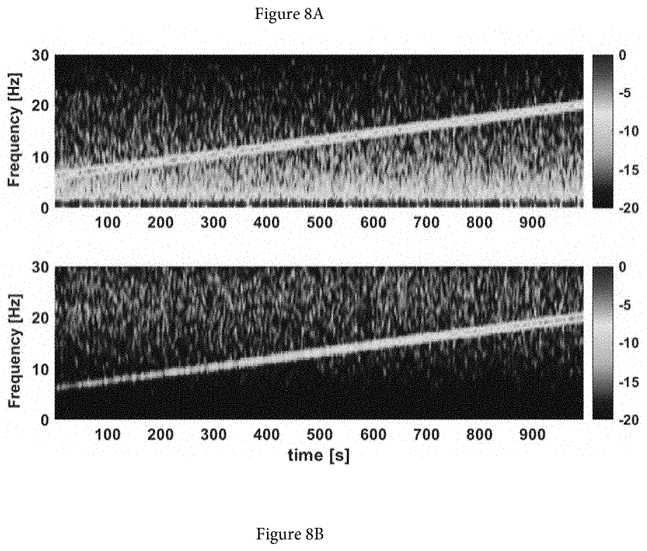

The exemplary graphs in FIGS. 6A-6C present, for example, the DSA derived from frontal EEG recorded from selected patients. The PSD and diffPSD were calculated for a 10-second EEG episode. For the PSD and diffPSD, the peak information was calculated using the setting with the excluded delta and extended alpha range. The examples provide a case for (i) both approaches detecting a peak (See, e.g., 6A), (ii) a peak detected for the diffPSD, but not the PSD approach (See, e.g., 6B), and (iii) ‘no peak’ in both approaches (See, e.g., 6C). The second case highlights the potential of the diffPSD to detect more subtle alpha oscillations.

FIGS. 6A-6C illustrate exemplary spectral diagrams and graphs of DSA (e.g., left) and PSD (e.g., right) for three exemplary cases with (See, e.g., FIG. 6A): Both, the PSD and the diffPSD approach detect a peak. The PSD was derived from second 4000 to 4010. (See, e.g., FIG. 6B). Only the diffPSD approach detects a peak; The PSD was derived from second 4000 to 4010. (See, e.g., FIG. 6C). No peak detected with either approach; The PSD was derived from second 1700 to 1710

### Exemplary Discussion

The exemplary diffEEG approach resulted in a more robust automated peak detection because the performance of diffPSD was not dependent on the range of excluded frequencies in the delta-range. Furthermore, the exemplary cases showed an optimized visualization of oscillatory alpha activity in the DSA and demonstrated the ability of the exemplary diffPSD to detect subtler alpha peaks than the exemplary PSD approach. To analyze these exemplary approaches, an interventional clinical trial was initiated, which investigated the influence of intraoperative frontal alpha maximization on patient outcome. (See, e.g., Reference 19). During general anesthesia with common substances like sevoflurane or propofol, EEG patterns with dominant oscillations in the delta and alpha frequency develop that give way to delta-dominant rhythms and ultimately EEG burst suppression. (See, e.g., References 23, 32, and 33). The state with alpha and delta rhythms can present a level of adequate anesthesia with thalamocortical oscillations in an idling state. (See, e.g., References 13, 23, and 32). Identification of strong delta oscillations in the raw EEG and the DSA can be straightforward since the DSA can present the delta oscillations in warmest colors because they can be the dominant frequency in the EEG. Strong oscillatory activity in the alpha range can be more difficult to track, especially when volatiles can be used as a maintenance anesthetic. These volatile anesthetics cause an increase in theta activity as well. (See, e.g., Reference 32). Because the exemplary system, method, and computer-accessible medium can be utilized to identify the highest dominant oscillatory activity, it can be beneficial for monitoring the current composition of brain electrical activity by means of the EEG.

The alpha oscillation can also serve as a marker for adequate analgesia management, because noxious stimulation can lead to a decrease in alpha power and bicoherence. (See, e.g., References 15 and 17). At the same time, age and/or cognitive impairments can change the characteristics of perioperatively detected alpha oscillations. (See, e.g., References 9, 23, and 34). For example, differences in oscillatory characteristics can help to evaluate the functional instead of the chronological age of the patient. (See, e.g., Reference 35). This correlation of frontal alpha oscillations with cognitive performance was not only shown in the perioperative setting, but also in the field of neurodegenerative diseases. (See, e.g., References 36 and 37). Therefore, the application of the exemplary diffPSD approach can be useful to diagnose dementia syndromes and to monitor disease progress. Other exemplary approaches for (alpha-) peak detection exist, for example, the frequently used linear regression. (See, e.g., References 7 and 10). The Fitting Oscillations & One-Over F (“FOOOF”) procedure for instance could help to identify oscillating components (See, e.g., Reference 25), but it needs significant computation. Thus, simpler differentiation procedures can be more usable and implementable to real-time monitoring systems. Furthermore, the centroid frequencies were calculated. These were significantly higher in the PSDs categorized as “no peak detected”. Therefore, the calculation of centroid frequency can be an additional exemplary parameter to assess the EEG and a validation tool for detected alpha peaks.

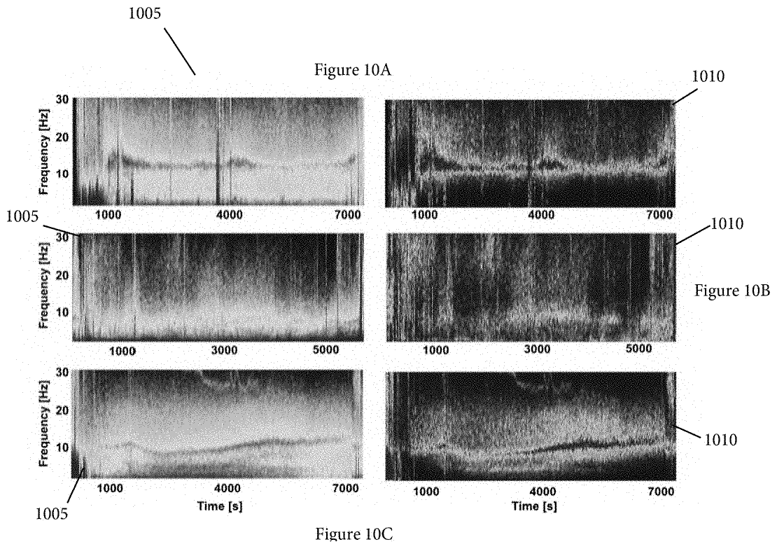

FIGS. 10A-10C show exemplary spectrograms according to an exemplary embodiment of the present disclosure. In particular, as illustrated in spectrograms in FIGS. 10A-10C examples of how the exemplary data processing method can improve visualization of alpha power. Spectrographs 1005 show EEG signals displayed as a digital spectral array in the typical method used on the most common intraoperative frontal EEG devices. The y-axis plots-frequency, the x-axis-time, and intense (e.g., “hot”) colors represent the magnitude of power (e.g., amplitude oscillation). At approximately 10 Hz the alpha power can be visualized. Spectrographs 1010 illustrate the same data displayed with the exemplary system, method, and computer-accessible medium. The alpha power can be easier to visually track—especially in FIGS. 10B and 10C (e.g., taken from elderly cases involving high dose sevoflurane).

The exemplary system, method and computer-accessible medium, according to an exemplary embodiment of the present disclosure, can be used to provide an indication to a medical professional (e.g., an anesthesiologist or nurse anesthetist) that a drop in the power spectra of the patient has been detected. The drop can be based on the normal alpha band for the patient, or the first derivative of the alpha band of the patient. For certain patients, for example, the signal from the normal alpha band can be strong enough to determine a power drop, and the indication can be provided based on this drop. However, in certain exemplary cases, the signal from the normal alpha band may not be strong enough. Thus, according to the exemplary embodiment of the present disclosure, the first derivative of the alpha band can be determined, and the exemplary indication can be provided based on the drop in the power spectra of the first derivative.

The exemplary indication provided to the medical professional can include an alarm or any other indication based on a predetermined drop in the power spectra. The exemplary alarm or exemplary indication can be visual, tactile and/or auditory, and can indicate to the medical professional that additional anesthesia medication should be provided to the patient. The predetermined drop can be set by the medical professional for each patient, or it can be fixed regardless of the patient. In some exemplary embodiments of the present disclosure, the predetermined drop can be about a 10% drop, a 15% drop, a 20% drop, a 25% drop, a drop therebetween, and/or any other suitable drop determined to be indicative of requiring additional anesthesia medication for the patient. All exemplary drops can be approximate, can vary, for example, by up to 30% of the value of the predetermined drop.

The exemplary system, method and computer-accessible medium can determine a baseline power spectra for the patient over a predetermined time series, which can be based on the approximate time of the medical procedure being performed on the patient. Once a baseline is determined, the predetermined drop can be based on the determined baseline. The baseline can also be obtained by taken initial measurements immediately before or after the administering of the anesthesia medication, and the drop can be determined based on this baseline. Additional, factors that can determine the exemplary drop can be the rate of the drop over time. For example, in one exemplary embodiment of the present disclosure, an indication may not be provided if there is a sudden drop in the power spectra, as such a sudden drop can be followed by an immediate increase. Thus, in such exemplary embodiment, if a sudden drop is detected, then the exemplary system, method and computer-accessible medium can wait a predetermined amount of time to determine if the power spectra increases before providing the indication. If the spectra does not increase in the predetermined amount of the time, then the indication can be provided. Additionally, if the drop occurs slowly over time, in a further exemplary embodiments of the present disclosure, then the indication can be provided immediately upon the detection of the predetermined amount of the drop.

Further, the exemplary system, method and computer-accessible medium, according to an exemplary embodiment of the present disclosure, can incorporate various machine learning procedures, such as neural networks (e.g., convolutional neural networks (“CNN”)), which can adjust the predetermined drop based on various patient factors. For example, an exemplary CNN can be used to analyze prior patient data and compare it to the date of the current patient. The exemplary system, method and computer-accessible medium, according to an exemplary embodiment of the present disclosure, can then provide a recommendation for the predetermined exemplary drop for the particular patient. Additionally, the exemplary system, method and computer-accessible medium, according to an exemplary embodiment of the present disclosure, can use the exemplary CNN to analyze and recommend potential anesthesia medication treatments plans (e.g., whether to increase or decrease certain types of anesthesia medication). Alternatively or in addition, the exemplary system, method and computer-accessible medium can, e.g., interface with an exemplary system for administering anesthesia, and can automatically increase or decrease the anesthesia medication provided to the patient based on the analysis of the alpha band or the first derivative of the alpha band.

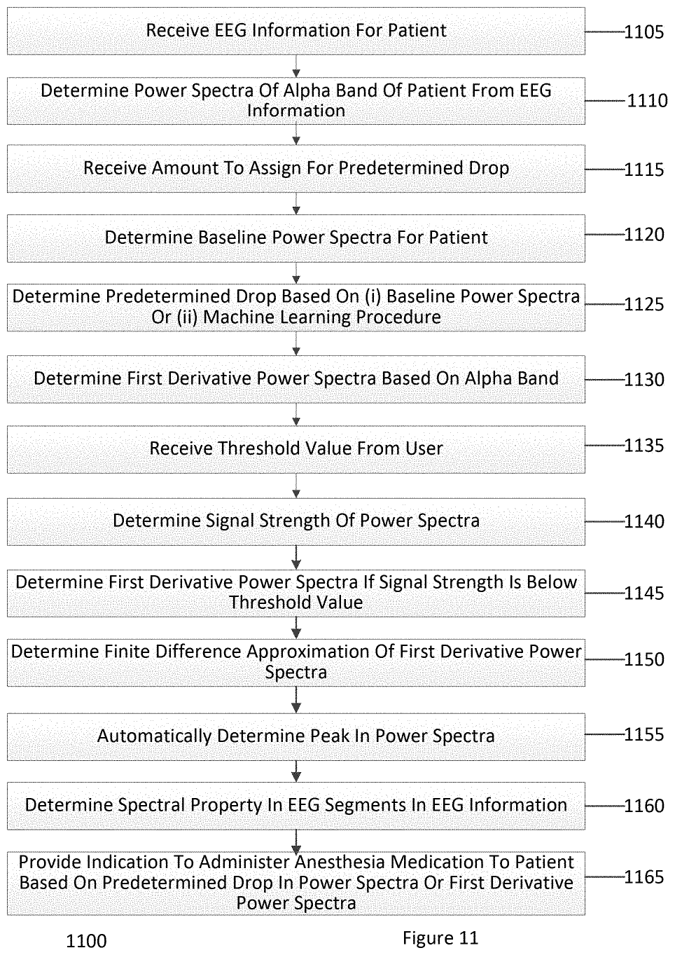

FIG. 11 shows an exemplary flow diagram of a method 1100 for providing an indication to administer an anesthesia medication to a patient according to an exemplary embodiment of the present disclosure. For example, at procedure 1105, EEG information for the patient can be received. At procedure 1110, a power spectra of an alpha band of the patient can be determined from the EEG information. At procedure 1115, an amount to assign for the predetermined drop can be received. At procedure 1120, a baseline power spectra for the patient can be determined. At procedure 1125, the predetermined drop can be determined based on (i) the baseline power spectra, and/or (ii) a machine learning procedure. At procedure 1130, a first derivative power spectra can be determined based on the alpha band. At procedure 1135, a threshold value can be received from a user. At procedure 1140, a signal strength of the power spectra can be determined. At procedure 1145, the first derivative power spectra can be determined if the signal strength is below a threshold value. At procedure 1150, a finite difference approximation of the first derivative power spectra can be determined. At procedure 1155, a peak in the power spectra can be automatically determined. At procedure 1160, a spectral property in EEG segments in the EEG information can be determined. At procedure 1165, an indication to administer the anesthesia medication to the patient can be provided based on a predetermined drop in the power spectra or the first derivative power spectra.

### Exemplary Use of the Exemplary System, Method, and Computer-Accessible

Medium for Sedation A feature common to patients admitted for intensive care can be the large sedation requirements necessary for synchronization with mechanical ventilation. During a pandemic, hospitals face critical shortages of many supplies including medications, innovative attempts to optimize care and manage resources without compromising patient safety are necessary.

Neurologic manifestations, including encephalopathy, can be under-recognized in ICU patients, and may result in an over-use of medications. During the height of the COVID-19 pandemic, it was determined that the most critically ill patients (e.g., 86%) receiving high dose sedation and/or neuromuscular blocking agents for ventilator synchrony during COVID infection exhibited patterns consistent with (i) low alpha power and first-derivative of alpha power, (ii) low total EEG power, and (iii) attenuated and discontinuous EEG patterns consistent with diffuse cerebral dysfunction and/or over-sedation. In the rare patients that did not exhibit discontinuous EEG, a more irregular EEG pattern (e.g., permutation entropy) was observed than might be expected based on the patient's age. When this information was analyzed, a reduction in sedative and analgesic requirements followed in 79% of patients without compromising patient care.

The exemplary system, method, and computer-accessible medium, according to an exemplary embodiment of the present disclosure, as described herein, can be used to determine how the frontal EEG in mechanically ventilated COVID+ patients can be utilized for titration of sedative medications. Exemplary sedative medications can include, but are not limited to, (i) Chloral hydrate, (ii) Midazolam, (iii) Pentobarbital, (iv) Fentanyl, (v) Ketamine, (vi) Precedex, (vii) Propofol, and (viii) Nitrous oxide.

Thirty patients admitted to intensive care units receiving mechanical ventilation for respiratory failure secondary to SARS-CoV-2 infection were included. Adhesive EEG electrodes (e.g., abbreviated montage—F1/2, F7/8, Fz) were placed over the forehead and expert EEG interpretation was provided to aid the ICU team in pharmacologic decision-making. Dose reductions of sedative, analgesic, and/or neuromuscular blocking agents within 24 hours of initiating frontal EEG monitoring were determined.

The majority of the EEG records demonstrated varying degrees of discontinuous, low-voltage activity consistent with high amounts of sedating medication. After sharing this information with the ICU providers, within 24 hours, most patients had significantly reduced sedation regimens.

Neurologic complications while receiving critical care, can delay extubation and can increase ICU stays. Managing ventilation synchrony in severely affected COVID+ patients can include an escalation of sedatives and/or neuromuscular blocking agents. In order to prevent over-use of medications already in critically short supply, an automatic analysis of frontal EEG patterns, using the exemplary system, method, and computer-accessible medium, can provide recommendations for pharmacologic decision-making.

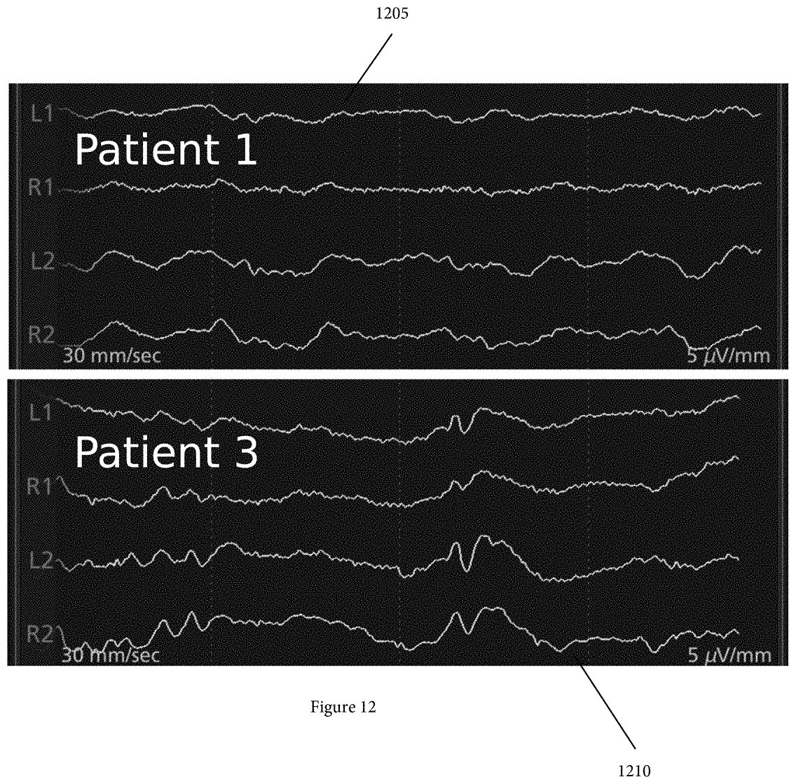

FIG. 12 shows a set of illustration of exemplary frontal EEG patterns of two patients illustrating varying degrees of discontinuous, low-voltage activity consistent with high amounts of sedating medication according to an exemplary embodiment of the present disclosure. Pattern 1205 illustrates severe attenuation of the frontal EEG signal while pattern 1210 illustrates discontinuous alternation between isoelectricity and low voltage/low frequency oscillations.

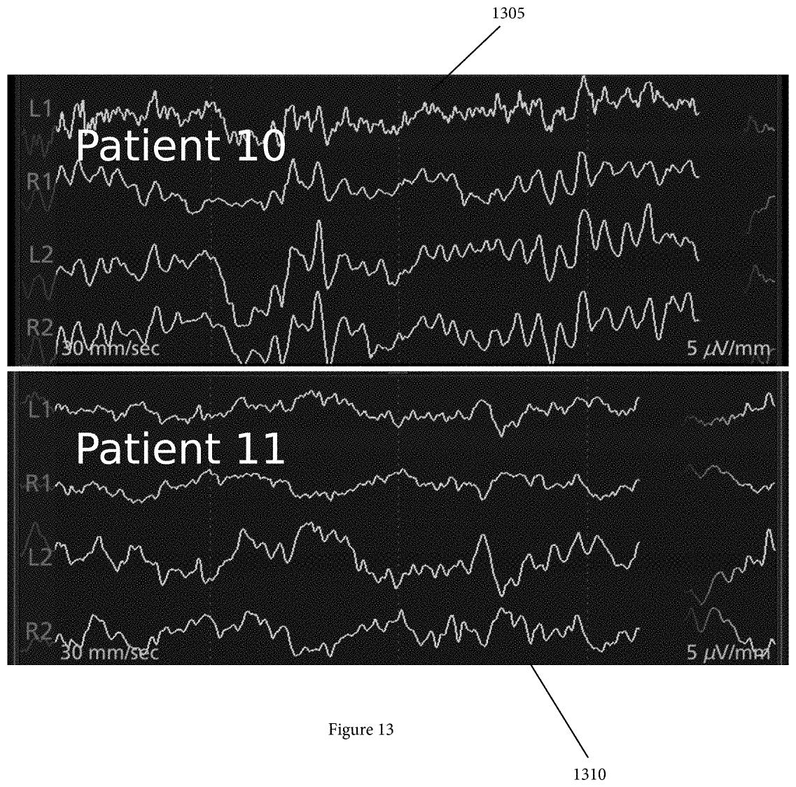

FIG. 13 shows a set of illustration of exemplary distinct frontal EEG patterns from two patients illustrating activity in the low and moderate frequency range according to an exemplary embodiment of the present disclosure. Pattern 1305 represents the EEG for a recently admitted patient who was intubated on the day of the EEG initiation. Pattern 1310 represents the EEG for a patient with an EEG initiated on Day 12 of mechanical ventilation, one day prior to trial extubation.

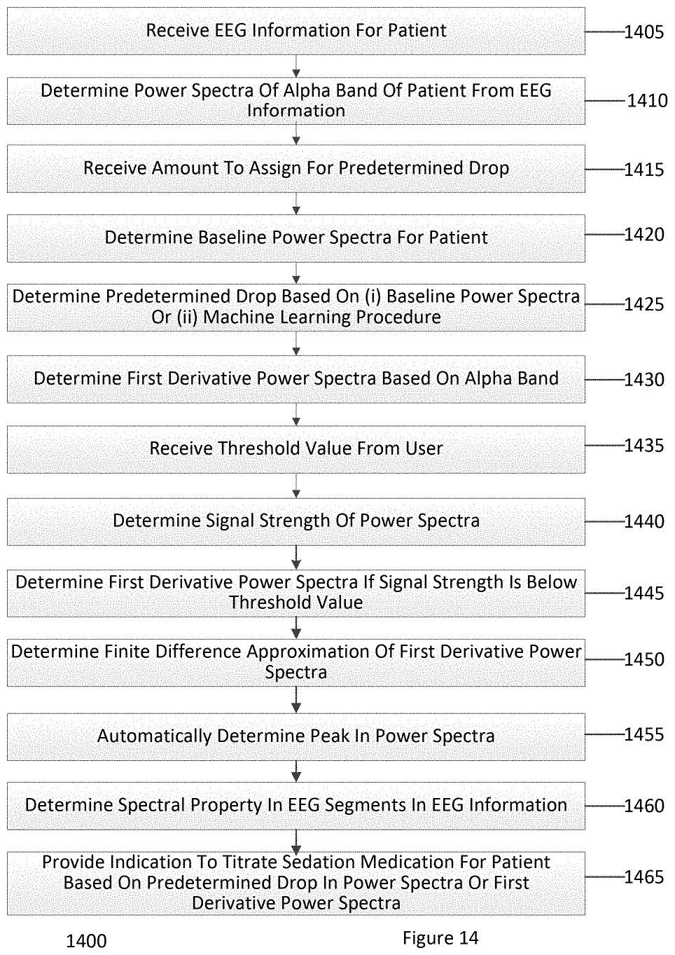

FIG. 14 shows an exemplary flow diagram of a method 1400 for providing an indication to titrate a sedation medication for a patient according to an exemplary embodiment of the present disclosure. For example, at procedure 1405, EEG information for the patient can be received. At procedure 1410, a power spectra of an alpha band of the patient can be determined from the EEG information. At procedure 1415, an amount to assign for the predetermined drop can be received. At procedure 1420, a baseline power spectra for the patient can be determined. At procedure 1425, the predetermined drop can be determined based on (i) the baseline power spectra, and/or (ii) a machine learning procedure. At procedure 1430, a first derivative power spectra can be determined based on the alpha band. At procedure 1435, a threshold value can be received from a user. At procedure 1440, a signal strength of the power spectra can be determined. At procedure 1445, the first derivative power spectra can be determined if the signal strength is below a threshold value. At procedure 1450, a finite difference approximation of the first derivative power spectra can be determined. At procedure 1455, a peak in the power spectra can be automatically determined. At procedure 1460, a spectral property in EEG segments in the EEG information can be determined. At procedure 1465, an indication to titrate the sedation medication for the patient can be provided based on a predetermined drop in the power spectra or the first derivative power spectra.

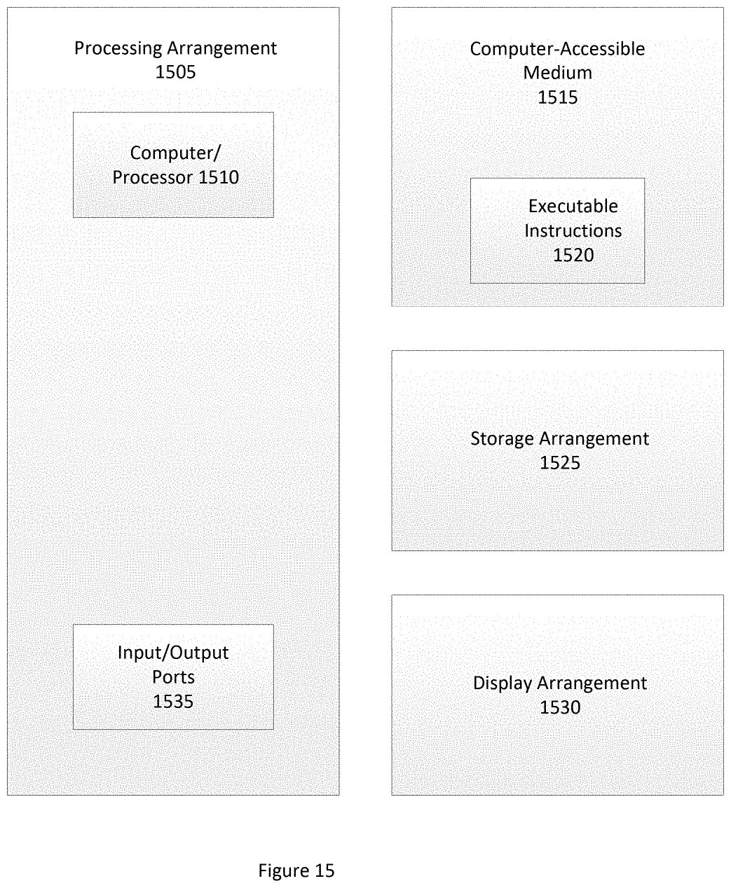

FIG. 15 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1505. Such processing/computing arrangement 1505 can be, for example entirely or a part of, or include, but not limited to, a computer/processor 1510 that can include, for example one or more microprocessors, and use instructions stored on a computer-accessible medium (e.g., RAM, ROM, hard drive, or other storage device).

As shown in FIG. 15, for example a computer-accessible medium 1515 (e.g., as described herein above, a storage device such as a hard disk, floppy disk, memory stick, CD-ROM, RAM, ROM, etc., or a collection thereof) can be provided (e.g., in communication with the processing arrangement 1505). The computer-accessible medium 1515 can contain executable instructions 1520 thereon. In addition or alternatively, a storage arrangement 1525 can be provided separately from the computer-accessible medium 1515, which can provide the instructions to the processing arrangement 1505 so as to configure the processing arrangement to execute certain exemplary procedures, processes, and methods, as described herein above, for example.

Further, the exemplary processing arrangement 1505 can be provided with or include an input/output ports 1535, which can include, for example a wired network, a wireless network, the internet, an intranet, a data collection probe, a sensor, etc. As shown in FIG. 15, the exemplary processing arrangement 1505 can be in communication with an exemplary display arrangement 1530, which, according to certain exemplary embodiments of the present disclosure, can be a touch-screen configured for inputting information to the processing arrangement in addition to outputting information from the processing arrangement, for example. Further, the exemplary display arrangement 1530 and/or a storage arrangement 1525 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties.
1. Aldecoa C, Bettelli G, Bilotta F, Sanders R D, Audisio R, Borozdina A, Cherubini A, Jones C, Kehlet H, MacLullich A, Radtke F, Riese F, Slooter A J, Veyckemans F, Kramer S, Neuner B, Weiss B, Spies C D. European Society of Anaesthesiology evidence-based and consensus-based guideline on postoperative delirium. Eur J Anaesthesiol 2017; 34:192-214.
2. Chan M T, Cheng B C, Lee T M, Gin T, Group C T. BIS-guided anesthesia decreases postoperative delirium and cognitive decline. J Neurosurg Anesthesiol 2013; 25:33-42.
3. Evered L, Silbert B, Knopman D S, Scott D A, DeKosky S T, Rasmussen L S, Oh E S, Crosby G, Berger M, Eckenhoff R G, Nomenclature Consensus Working G. Recommendations for the nomenclature of cognitive change associated with anaesthesia and surgery-2018. Br J Anaesth 2018; 121:1005-12.
4. Radtke F M, Franck M, Lendner J, Kruger S, Wemecke K D, Spies C D. Monitoring depth of anaesthesia in a randomized trial decreases the rate of postoperative delirium but not postoperative cognitive dysfunction. Br J Anaesth 2013; 110 Suppl 1:i98-105.
5. Hesse S, Kreuzer M, Hight D, Gaskell A, Devari P, Singh D, Taylor N B, Whalin M K, Lee S, Sleigh J W, Garcia P S. Association of electroencephalogram trajectories during emergence from anaesthesia with delirium in the post-anaesthesia care unit: an early sign of postoperative complications. Br J Anaesth 2019; 122:622-34.
6. A glossary of terms most commonly used by clinical electroencephalographers. Electroencephalogr Clin Neurophysiol 1974; 37:538-48.
7. Hight D, Voss L J, Garcia P S, Sleigh J. Changes in Alpha Frequency and Power of the Electroencephalogram during Volatile-Based General Anesthesia. Front Syst Neurosci 2017; 11:36.
8. Purdon P L, Pierce E T, Mukamel E A, Prerau M J, Walsh J L, Wong K F, Salazar-Gomez A F, Harrell P G, Sampson A L, Cimenser A, Ching S, Kopell N J, Tavares-Stoeckel C, Habeeb K, Merhar R, Brown E N. Electroencephalogram signatures of loss and recovery of consciousness from propofol. Proc Natl Acad Sci USA 2013; 110:E1142-51.
9. Schultz A, Grouven U, Zander I, Beger F A, Siedenberg M, Schultz B. Age-related effects in the EEG during propofol anaesthesia. Acta Anaesthesiol Scand 2004; 48:27-34.
10. Sleigh J W, Leslie K, Voss L. The effect of skin incision on the electroencephalogram during general anesthesia maintained with propofol or desflurane. J Clin Monit Comput 2010; 24:307-18.
11. Ching S, Cimenser A, Purdon P L, Brown E N, Kopell N J. Thalamocortical model for a propofol-induced alpha-rhythm associated with loss of consciousness. Proc Natl Acad Sci USA 2010; 107:22665-70.
12. Liley D, Bojak I, Dafilis M P, van Veen L, Frascoli F, Foster B. Bifurcations and state changes in the human alpha rhythm: Theory and experiment, 2010:117-45.
13. Franks N P. General anaesthesia: from molecular targets to neuronal pathways of sleep and arousal. Nat Rev Neurosci 2008; 9:370-86.
14. Feshchenko V A, Veselis R A, Reinsel R A. Propofol-induced alpha rhythm. Neuropsychobiology 2004; 50:257-66.
15. Hagihira S, Takashina M, Mori T, Ueyama H, Mashimo T. Electroencephalographic bicoherence is sensitive to noxious stimuli during isoflurane or sevoflurane anesthesia. Anesthesiology 2004; 100:818-25.
16. Kochs E, Bischoff P, Pichlmeier U, Schulte am Esch J. Surgical stimulation induces changes in brain electrical activity during isoflurane/nitrous oxide anesthesia. A topographic electroencephalographic analysis. Anesthesiology 1994; 80:1026-34.
17. Hight D F, Gaskell A L, Kreuzer M, Voss L J, Garcia P S, Sleigh J W. Transient electroencephalographic alpha power loss during maintenance of general anaesthesia. Br J Anaesth 2019.
18. Kreuzer M. EEG Based Monitoring of General Anesthesia: Taking the Next Steps. Front Comput Neurosci 2017; 11:56.
19. Gaskell A, Pullon R, Hight D, Termaat J, Mans G, Voss L, Kreuzer M, Schmid S, Kratzer S, Rodriguez A, Schneider G, Garcia P, Sleigh J. Modulation of frontal EEG alpha oscillations during maintenance and emergence phases of general anaesthesia to improve early neurocognitive recovery in older patients: protocol for a randomised controlled trial. Trials 2019; 20:146.
20. Koch S, Spies C. Neuromonitoring in the elderly. Curr Opin Anaesthesiol 2019; 32:101-7.
21. Dijk D J, Beersma D G, van den Hoofdakker R H. All night spectral analysis of EEG sleep in young adult and middle-aged male subjects. Neurobiol Aging 1989; 10:677-82.
22. United Nations. World Population Prospects The 2010 Revision, 2011.
23. Purdon P L, Pavone K J, Akeju O, Smith A C, Sampson A L, Lee J, Zhou D W, Solt K, Brown E N. The Ageing Brain: Age-dependent changes in the electroencephalogram during propofol and sevoflurane general anaesthesia. Br J Anaesth 2015; 115 Suppl 1:i46-i57.
24. Colombo M A, Napolitani M, Boly M, Gosseries O, Casarotto S, Rosanova M, Brichant J-F, Boveroux P, Rex S, Laureys S, Massimini M, Chieregato A, Sarasso S. The spectral exponent of the resting EEG indexes the presence of consciousness during unresponsiveness induced by propofol, xenon, and ketamine. Neuroimage 2019; 189:631-44.
25. Haller M, Donoghue T, Peterson E, Varma P, Sebastian P, Gao R, Noto T, Knight R T, Shestyuk A, Voytek B. Parameterizing neural power spectra. bioRxiv 2018:299859.
26. Wilmott P, Howson S, Howison S, Dewynne J. The mathematics of financial derivatives: a student introduction: Cambridge university press, 1995.
27. Burch N R, Nettleton W J, Sweeney J, Edwards R. Period analysis of the electroencephalogram on a general-purpose digital computer. Ann N Y Acad Sci 1964; 115:827-43.
28. Kedem B. Spectral analysis and discrimination by zero-crossings. Proceedings of the IEEE 1986; 74:1477-93.
29. Iglewicz B, Hoaglin D C. How to detect and handle outliers Milwaukee, Wis.: American Society for Quality Control, 1993.
30. Hentschke H, Stuttgen M C. Computation of measures of effect size for neuroscience data sets. Eur J Neurosci 2011; 34:1887-94.
31. Amrhein V, Greenland S, McShane B. Scientists rise up against statistical significance. Nature 2019; 567:305-7.
32. Akeju O, Westover M B, Pavone K J, Sampson A L, Hartnack K E, Brown E N, Purdon P L. Effects of sevoflurane and propofol on frontal electroencephalogram power and coherence. Anesthesiology 2014; 121:990-8.
33. Brown E N, Lydic R, Schiff N D. General anesthesia, sleep, and coma. N Engl J Med 2010; 363:2638-50.
34. Giattino C M, Gardner J E, Sbahi F M, Roberts K C, Cooter M, Moretti E, Browndyke J N, Mathew J P, Woldorff M G, Berger M, Investigators M-P. Intraoperative Frontal Alpha-Band Power Correlates with Preoperative Neurocognitive Function in Older Adults. Front Syst Neurosci 2017; 11:24.
35. Kreuzer M, Whalin M K, Hesse S D W, Riso M A, Garcia P S. Anesthetic Management of a Patient With Multiple Previous Episodes of Postanesthesia Care Unit Delirium: A Case Report. A A Case Rep 2017; 8:311-5.
36. van der Zande J J, Gouw A A, van Steenoven I, Scheltens P, Stan C J, Lemstra A W. EEG Characteristics of Dementia With Lewy Bodies, Alzheimer's Disease and Mixed Pathology. Front Aging Neurosci 2018; 10:190.
37. Arakaki X, Lee R, King K S, Fonteh A N, Harrington M G. Alpha desynchronization during simple working memory unmasks pathological aging in cognitively healthy individuals. PLoS One 2019; 14:e0208517.
38. Akeju O, Song A H, Hamilos A E, Pavone K J, Flores F J, Brown E N, Purdon P L. Electroencephalogram signatures of ketamine anesthesia-induced unconsciousness. Clin Neurophysiol 2016; 127:2414-22.

1. A non-transitory computer-accessible medium having stored thereon computer-executable instructions for providing at least one indication to administer at least one anesthesia medication to at least one patient, wherein, when a computing arrangement executes the instructions, the computing arrangement is configured to perform procedures comprising:
receiving electroencephalogram (EEG) information for the at least one patient;
determining at least one power spectra of an alpha band of the at least one patient from the EEG information; and
providing the at least one indication to administer the at least one anesthesia medication to the at least one patient based on a predetermined drop in the at least one power spectra.

2. (canceled)

3. The computer-accessible medium of claim 1, wherein the predetermined drop is one of (i) about 10%, (ii) about 15%, (iii) about 20%, or iv) about 25%.

4. The computer-accessible medium of claim 1, wherein the computer arrangement is further configured to receive an amount to assign for the predetermined drop.

5. The computer-accessible medium of claim 1, wherein the computer arrangement is further configured to determine a baseline power spectra for the at least one patient.

6. The computer-accessible medium of claim 5, wherein the computer arrangement is configured to determine the baseline power spectra over a predetermined time series.

7. The computer-accessible medium of claim 6, wherein the predetermined time series is based on an approximate time of a medical procedure to be performed on the at least one patient.

8. The computer-accessible medium of claim 6, wherein the computer arrangement is configured to determine the predetermined drop based on the baseline power spectra.

9. The computer-accessible medium of claim 8, wherein the computer arrangement is configured to determine the predetermined drop based on a rate of a drop in the at least one power spectra over time.

10. The computer-accessible medium of claim 1, wherein the computer arrangement is configured to determine the predetermined drop using at least one machine learning procedure.

11. The computer-accessible medium of claim 10, wherein the at least one machine learning procedure is a convolutional neural network.

12. The computer-accessible medium of claim 1, wherein the computer arrangement is further configured to:
determine a first derivative power spectra based on the alpha band; and
provide the at least one indication to administer the at least one anesthesia medication to the at least one patient based on a further predetermined drop in the first derivative power spectra.

13. The computer-accessible medium of claim 12, wherein the computer arrangement is further configured to:
determine a signal strength of the at least one power spectra; and
determine the first derivative power spectra if the signal strength is below a threshold value.

14. The computer-accessible medium of claim 13, wherein the computer arrangement is configured to receive the threshold value from at least one user.

15. The computer-accessible medium of claim 12, wherein the computer arrangement is further configured to determine a finite difference approximation of the first derivative power spectra.

16. The computer-accessible medium of claim 1, wherein the computer arrangement is further configured to automatically determine at least one peak in the at least one power spectra.

17. The computer-accessible medium of claim 16, wherein the computer arrangement is configured to automatically determine the at least one peak using a linear regression procedure.

18. The computer-accessible medium of claim 1, wherein the computer arrangement is further configured to determine at least one spectral property in EEG segments in the EEG information.

19. A method for providing at least one indication to administer at least one anesthesia medication to at least one patient, comprising:
receiving electroencephalogram (EEG) information for the at least one patient;
determining at least one power spectra of an alpha band of the at least one patient from the EEG information; and
using a computer hardware arrangement, providing the at least one indication to administer the at least one anesthesia medication to the at least one patient based on a predetermined drop in the at least one power spectra.

20-36. (canceled)

37. A system for providing at least one indication to administer at least one anesthesia medication to at least one patient, comprising:
a computer hardware arrangement configured to:
receive electroencephalogram (EEG) information for the at least one patient;
determine at least one power spectra of an alpha band of the at least one patient from the EEG information; and
provide the at least one indication to administer the at least one anesthesia medication to the at least one patient based on a predetermined drop in the at least one power spectra.

38-54. (canceled)

55. A non-transitory computer-accessible medium having stored thereon computer-executable instructions for providing at least one indication to titrate at least one sedation medication for at least one patient, wherein, when a computing arrangement executes the instructions, the computing arrangement is configured to perform procedures comprising:
receiving electroencephalogram (EEG) information for the at least one patient;
determining at least one power spectra of an alpha band of the at least one patient from the EEG information; and
providing the at least one indication to titrate the at least one sedation medication for the at least one patient based on a predetermined drop in the at least one power spectra.

56-71. (canceled)

72. A method for providing at least one indication to titrate at least one sedation medication for at least one patient, comprising:
receiving electroencephalogram (EEG) information for the at least one patient;
determining at least one power spectra of an alpha band of the at least one patient from the EEG information; and
using a computer hardware arrangement, providing the at least one indication to titrate the at least one sedation medication for the at least one patient based on a predetermined drop in the at least one power spectra.

73-88. (canceled)

89. A system for providing at least one indication to titrate at least one sedation medication for at least one patient, comprising:
a computer hardware arrangement configured to:
receive electroencephalogram (EEG) information for the at least one patient;
determine at least one power spectra of an alpha band of the at least one patient from the EEG information; and
provide the at least one indication to titrate the at least one sedation medication for the at least one patient based on a predetermined drop in the at least one power spectra.

90-105. (canceled)
